(19) United States
(12) Patent Application Publication
(10) Pub. No.: US 2005/0026290 A1
(43) Pub. Date: Feb. 3, 2005

(54) Inhibiting gene expression with dsRNA

(76) Inventors: Joseph Anthony Ciardi, Philadelphia, PA (US); Kenichi Shibuya, Gainesville, FL (US); Holly Loucas, Newberry, FL (US); David Clark, Gainesville, FL (US)

Correspondence Address: Dr. O. M. (Sam) Zaghmout;Bio Intellectual Property Services (BIO IPS), 8509 Kernon CT, Lorton, VA, 22079, US

(21) Appl. No.: 10/631,718

(22) Filed: Aug. 1, 2003

(51) Int. Cl. A01H 1/00; C12N 15/82

(52) U.S. Cl. 435/468; 800/287

(57) ABSTRACT

The present invention relates to the altering or controlling inhibition of gene expression in a cell using a methodology involving a dsRNA. The use of the method in plants and plant cells has been found to be particularly beneficial. For example the inhibition of a hormone signaling gene in a plant, such as the EIN2 gene, permits the modulating of flower longevity in the selected plant. This is accomplished by using our RNAi technology in inhibiting the expression of an ethylene-modulated gene or an ethylene signaling gene in plants and plant cells. This method provides for the use of RNAi technology in inhibiting the expression of a hormone signaling gene in a plant, particularly in the floral parts of a plant. Thus, one can modulate flower longevity in a plant.

### FIELD OF THE INVENTION

The present invention relates to methods, compositions and kits for inhibiting gene expression in a cell. The particular technique disclosed and claimed calls for the use of double stranded RNA (dsRNA) for controlling and/or inhibiting gene expression where such control is regarded as desirable. In particular, the use of this new technique is described for use in inhibiting gene expression in plant cells and plants using dsRNA.

### BACKGROUND

The information provided below is not admitted to be prior art to the present invention, but is provided solely to assist the understanding of the reader.

The benefits of being able to inhibit the expression of a specific gene or group of genes in a cell such as, but not limited to, a plant cell are obvious. Many undesirable phenotypes arise from the expression of a particular gene or group of genes that are regulated or likely to be regulated by compounds, such as ethylene. The inhibition of the gene or group of genes which give rise to undesirable phenotypes can therefore be accomplished by regulating the compound generating gene, for example, the ethylene-modulated gene(s) in a cell. Such undesirable phenotypes can also result from the expression of a mutant form of protein. In that case it would be advantageous to eliminate the expression of this type of mutant allele. In addition, such gene specific inhibition may be used to increase flower longevity, increase leaf abscission, regulate fruit ripening and so on.

In addition, the elimination or inhibition of expression of a specific gene can be used to study and manipulate developmental events in a cell (e.g., flower cell). For example, valuable information could be obtained if the function of the gene of interest could be disturbed in specific cells of the flower and at defined times. In such a situation, the classical techniques of gene “knockout” cannot be used, because they eliminate gene function universally throughout the flower. Existing “knockout” technology is extremely laborious. It necessitates first making a disrupted gene segment that is suitably marked to enable the selection of homologous recombination events in cultured cells. Such cells must then be regenerated and the resulting chimeric plants used to establish pure breeding lines before homozygous mutants can be obtained.

Antisense technology has been the most commonly described approach in protocols to achieve gene-specific interference. For antisense strategies, stochiometric amounts of single-stranded nucleic acid complementary to the messenger RNA for the gene of interest are introduced into the cell. Some difficulties with antisense-based approaches relate to delivery, stability, and dose requirements. In general, cells do not have an uptake mechanism for single-stranded nucleic acids. Hence uptake of unmodified single-stranded material is extremely inefficient. While waiting for uptake into cells, the single-stranded material is subject to degradation. Because antisense interference requires that the interfering material accumulate at a relatively high concentration (at or above the concentration of endogenous mRNA), the amount required to be delivered is a major constraint on efficacy. As a consequence, much of the effort in developing antisense technology has been focused on the production of modified nucleic acids that are both stable to nuclease digestion and able to diffuse readily into cells. The use of antisense interference for gene therapy or other whole-organism applications has been limited by the large amounts of oligonucleotide that need to be synthesized from non-natural analogs, the cost of such synthesis, and the difficulty even with high doses of maintaining a sufficiently concentrated and uniform pool of interfering material in each cell.

Another method for engineered interference is based on a triple helical nucleic acid structure. This approach relies on the rare ability of certain nucleic acid populations to adopt a triple-stranded structure. Under physiological conditions, nucleic acids are virtually all single- or double-stranded, and rarely if ever form triple-stranded structures. It has been known for some time, however, that certain simple purine- or pyrimidine-rich sequences could form a triple-stranded molecule in vitro under extreme conditions of pH (i.e., in a test tube). Such structures are generally very transient under physiological conditions, so that simple delivery of unmodified nucleic acids designed to produce triple-strand structures does not yield interference. As with antisense, development of triple-strand technology for use in vivo has focused on the development of modified nucleic acids that would be more stable and more readily absorbed by cells in vivo. An additional goal in developing this technology has been to produce modified nucleic acids for which the formation of triple-stranded material proceeds effectively at physiological pH.

Gene expression has been shown to be reduced by Triple-Helix Structures. Triple-strand structures occur rarely, if at all, under physiological conditions and are limited to very unusual base sequence with long runs of purines and pyrimidines. By contrast, dsRNA-mediated inhibition occurs efficiently under physiological conditions, and occurs with a wide variety of inhibitory and target nucleotide sequences. The present invention has been used to inhibit expression of an ethyelene-signaling gene.

Another approach to gene-specific interference is a set of operational procedures grouped under the name “co-suppression”. This approach was first described in plants and refers to the ability of transgenes to cause silencing of an unlinked but homologous gene. More recently, phenomena similar to co-suppression have been reported in two animals: C. elegans and Drosophila. Co-suppression was first observed by accident, with reports coming from groups using transgenes in attempts to achieve over-expression of a potentially useful locus. In some cases the over-expression was successful while, in many others, the result was opposite from that expected. In those cases, the transgenic plants actually showed less expression of the endogenous gene. Several mechanisms have so far been proposed for transgene-mediated co-suppression in plants; all of these mechanistic proposals remain hypothetical, and no definitive mechanistic description of the process has been presented. The models that have been proposed to explain co-suppression can be placed in two different categories. In one set of proposals, a direct physical interaction at the DNA- or chromatin-level between two different chromosomal sites has been hypothesized to occur; an as-yet-unidentified mechanism would then lead to de novo methylation and subsequent suppression of gene expression. Alternatively, some have postulated an RNA intermediate, synthesized at the transgene locus, which might then act to produce interference with the endogenous gene. The characteristics of the interfering RNA, as well as the nature of the interference process, have not been determined. Recently, a set of experiments with RNA viruses have provided some support for the possibility of RNA intermediates in the interference process. In these experiments, a replicating RNA virus is modified to include a segment from a gene of interest. This modified virus is then tested for its ability to interfere with expression of the endogenous gene. Initial results with this technique have been encouraging. However, the properties of the viral RNA that are responsible for the observed interference effects have not been determined and, in any case, would be limited to plants which are hosts of the plant virus.

Gene expression has also been reported to be reduced by transgene-mediated genetic interference phenomenon called co-suppression which may include a wide variety of different processes. A confounding aspect in creating a general technique based on co-suppression is that some transgenes in plants lead to suppression of the endogenous locus and some do not. A sense suppression of a chalcone synthase gene in Petunia results in flowers with altered pigmentation and antisense suppression of a polygalacturonidase gene in tomato leads to delayed fruit ripening. Results in C. elegans and Drosophila indicate that certain transgenes can cause interference (i.e., a quantitative decrease in the activity of the corresponding endogenous locus) but that most transgenes do not produce such an effect. Unfortunately, these methods are often variable and unpredictable in their ability to alter gene expression, and in many cases a complete disruption of the particular gene activity is not achieved. The lack of a predictable effect in plants, nematodes, and insects greatly limits the usefulness of simply adding transgenes to the genome to interfere with gene expression. Viral-mediated co-suppression in plants appears to be quite effective, but has a number of drawbacks. For example, it is not clear what aspects of the viral structure are critical for the observed interference.

The present invention differs from antisense-mediated interference in both approach and effectiveness. Antisense-mediated genetic interference methods have a major challenge: delivery to the interior of the cell of specific single-stranded nucleic acid molecules at a concentration that is equal to or greater than the concentration of endogenous mRNA. Double-stranded RNA-mediated inhibition has advantages both in the stability of the material to be delivered and the concentration required for effective inhibition. Below, we disclose that in the model petunia, in the present invention, is at least 30-fold more effective than an equivalent antisense approach (i.e., dsRNA is at least 30-fold more effective than the antisense-transformation mediated interference in reducing gene expression). These comparisons also suggest that inhibition of gene expression by double-stranded RNA must occur by a mechanism distinct from antisense interference.

Other known methods used to alter gene expression within a cell include the use of catalytic ribonucleotides or ribozymes. These can be technically challenging. Similarly, homologous gene disruption, although the most desirable genetically, is unfortunately not efficient enough with currently available techniques to be routinely used for such purposes.

Thus, there is a long-felt but unfulfilled need for a novel method which would permit one to effectively and predictably alter the expression of a gene in cells, in general, and particularly in plant cells in order to obtain plants with improved and commercially important properties.

The present invention avoids the disadvantages of the previously-described methods for genetic interference. Several advantages of the present invention are discussed below, but numerous others will be apparent to one of ordinary skill in the biotechnology and genetic engineering arts.

### SUMMARY OF THE INVENTION

The present invention relates to the altering or controlling inhibition of gene expression in a cell using a methodology involving a dsRNA. The use of the method in plants and plant cells has been found to be particularly beneficial. For example the inhibition of a hormone signaling gene in a plant, such as the EIN2 gene, permits the modulating of flower longevity in the selected plant. This is accomplished by using our RNAi technology in inhibiting the expression of an ethylene-modulated gene in plants and plant cells. This method provides for the use of RNAi technology in inhibiting the expression of a hormone signaling gene in a plant, particularly in the floral parts of a plant. Thus, one can modulate flower longevity in a plant.

The present invention, also, relates to the production of plants with improved properties and traits using molecular techniques and genetic transformation. In particular, the invention relates to methods of altering the expression of a gene in a plant cell using sense and antisense RNA fragments of the target gene. Importantly, such sense and antisense RNA fragments are capable of forming a double-stranded RNA molecule.

The invention also relates to plant cells obtained using such methods, to plants derived from such cells, to the progeny of such plants and to seeds derived from such plants. In such plant cells or plants, the alteration of the gene expression of a particular gene is more effective, selective and more predictable than the alteration of the gene expression of a particular gene obtained using current methods known in the art.

Generally, it is the object of the present invention to provide a method for inhibiting the expression of a target gene in a cell, such as a plant cell. The method comprising: introducing into the cell an RNA comprising a double stranded structure having a nucleotide sequence which is substantially identical to at least a part of the target gene, wherein the wherein the target gene is an ethylene signaling gene or is likely to be regulated by ethylene.

The invention therefore provides a method comprising introducing into a plant cell a sense RNA fragment of a target gene and an antisense RNA fragment of said target gene, wherein said sense RNA fragment and said antisense RNA fragment are capable of forming a double-stranded RNA molecule, wherein the expression of said target gene in said cell is altered. In one embodiment, the RNA fragments are comprised in two different RNA molecules. In another embodiment, the RNA fragments are mixed before being introduced into said cell.

In another embodiment, the RNA fragments are mixed before being introduced into said cell under conditions allowing them to form a double-stranded RNA molecule. In another embodiment, the RNA fragments are introduced into said cell sequentially. In yet another preferred embodiment, the RNA fragments are comprised in one RNA molecule. In such case, the RNA molecule is preferably capable of folding such that said RNA fragments comprised therein form a double-stranded RNA molecule.

In another embodiment, the target gene is a native gene of a plant cell, preferably an essential gene of said plant cell. The invention also further provides a plant cell comprising the sense and antisense RNA fragments of the present invention, wherein the expression of said target gene in said plant cell is altered by said RNA fragments, a plant and the progeny thereof derived from the plant cell, propagation materials and seeds derived from the plant.

In yet another embodiment, methods such as aptazymes, maxizymes and intramers can potentially find a use in modulating gene function at the mRNA or proteins. These methods were reviewed recently by Famulok et al (Trends in Biotechnology. 2002. November issue. Volume: 20, Issue 11, Pages: 462-466).

Definitions:

Unless defined otherwise, all technical and scientific terms used herein have the same meaning as commonly understood by one of ordinary skill in the art to which this invention belongs. Still, certain elements are defined below for the sake of clarity and ease of reference.

An “siRNA” or “RNAI” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferably about 20-30 base nucleotides, more preferably about 20-25 nucleotides in length.

“Antiparallel” refers to two nucleotide sequences paired through hydrogen bonds between complementary base residues with phosphodiester bonds running in the 5′-3′ direction in one nucleotide sequence and in the 3′-5′ direction in the other nucleotide sequence.

“Associated With/Operatively Linked” refers to two DNA sequences that are related physically or functionally linked. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

“Chimeric Gene” refers to a recombinant DNA sequence in which a promoter or regulatory DNA sequence is operatively linked to, or associated with, a DNA sequence that codes for an mRNA or which is expressed as a protein, such that the regulator DNA sequence is able to regulate transcription or expression of the associated DNA sequence. The regulator DNA sequence of the chimeric gene is not normally operatively linked to the associated DNA sequence as found in nature.

“Coding Sequence” refers to a nucleic acid sequence that is transcribed into RNA such as nRNA, rRNA, tRNA, snRNA, sense RNA or antisense RNA. Preferably the RNA is then translated in an organism to produce a protein.

“Complementary” refers to two nucleotide sequences which comprise antiparallel nucleotide sequences capable of pairing with one another upon formation of hydrogen bonds between the complementary base residues in the antiparallel nucleotide sequences.

By “comprising” is meant including, but not limited to, whatever follows the word “comprising”. Thus, use of the term “comprising” indicates that the listed elements are required or mandatory, but that other elements are optional and may or may not be present. By “consisting of ” is meant including, and limited to, whatever follows the phrase “consisting of ”. Thus, the phrase “consisting of ” indicates that the listed elements are required or mandatory, and that no other elements may be present. By “consisting essentially of ” is meant including any elements listed after the phrase, and limited to other elements that do not interfere with or contribute to the activity or action specified in the disclosure for the listed elements. Thus, the phrase “consisting essentially of ” indicates that the listed elements are required or mandatory, but that other elements are optional and may or may not be present depending upon whether or not they affect the activity or action of the listed elements.

“Expression” refers to the transcription and/or translation of an endogenous gene or a transgene in plants or the cells of plants. In the case of antisense constructs, for example, expression may refer to the transcription of the antisense DNA only.

“Gene” refers to a defined region that is located within a genome and that, besides the aforementioned coding nucleic acid sequence, comprises other, primarily regulatory, nucleic acid sequences responsible for the control of expression, i.e., transcription and translation of the coding portion. A gene may also comprise other 5′ and 3′ untranslated sequences and termination sequences. Further elements that may be present are, for example, introns.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms, also, includes non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

“Homologous DNA Sequence” refers to a DNA sequence naturally associated with a host cell into which it is introduced.

A “target gene” is any gene in a cell. For example, a target gene is a gene of known function or is a gene whose function is unknown, but whose total or partial nucleotide sequence is known. Alternatively, the function of a target gene and its nucleotide sequence are both unknown. A target gene is a native gene of the plant cell or is a heterologous gene which had previously been introduced into the plant cell or a parent cell of said plant cell, for example by genetic transformation. A heterologous target gene is stably integrated in the genome of the plant cell or is present in the plant cell as an extrachromosomal molecule, e.g. as an autonomously replicating extrachromosomal molecule.

“Native” refers to a gene that is present in the genome of an untransformed cell.

“Naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

An “essential” gene is a gene encoding a protein such as a biosynthetic enzyme, receptor, signal transduction protein, structural gene product, or transport protein that is essential to the growth or survival of the plant.

EIN refers to a subset of the genes involved in ethylene signaling and include, but not limited to, EIN2, EIN3, EIN4, EIN5, and EIN7. EIN genes may fall into these categories: 1. Genes involved in plant development 2. Members of signal transduction pathways 3. Hormone signaling genes 4. Ethylene signaling genes.

Ethylene Signaling gene refers to, but not limited to, any genes involved in ethylene signal transduction including receptor genes (ETR1, ETR2, ERS1, ERS2, EIN4), CTR1, EIN2, EIN3, EIL1, EIL2, EIL3, EIL4, EIL5, RAN1, and ERF1. Ethylene Signaling genes may fall into these categories: 1. genes involved in plant development 2. members of signal transduction pathways 3. hormone signaling genes.

To “alter” the expression of a target gene in a plant cell means that the level of expression of the target gene in a plant cell after applying a method of the present invention is different from its expression in the cell before applying the method. To alter gene expression preferably means that the expression of the target gene in the plant is reduced, preferably strongly reduced, more preferably the expression of the gene is not detectable. The alteration of the expression of an essential gene may result in a knockout mutant phenotype in plant cells or plants derived therefrom.

“Isolated nucleic acid” is, in the context of the present invention, an isolated nucleic acid molecule that, by the hand of man, exists apart from its native environment and is therefore not a product of nature. An isolated nucleic acid molecule may exist in a purified form or may exist in a non-native environment such as, for example, a transgenic host cell.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue or organ or stage of development.

“Heterologous” as used herein means “of different natural origin” or represents a non-natural state. For example, if a host cell is transformed with a nucleic acid sequence derived from another organism, particularly from another species, that nucleic acid sequence is heterologous with respect to that host cell and also with respect to descendants of the host cell which carry that nucleic acid sequence. Similarly, heterologous refers to a nucleotide sequence derived from and inserted into the same natural, original cell type, but which is present in a non-natural state, e.g. a different copy number, or under the control of different regulatory elements.

A “knock-out” of a gene means an alteration in the sequence of the gene that results in a decrease of function of the target gene, preferably such that target gene expression is undetectable or insignificant. Transgenic knock-out plants can be comprise a heterozygous knock-out of a target gene, or a homozygous knock-out of a target gene.

By “operably linked” is meant that a DNA sequence and a regulatory sequence(s) are connected in such a way as to permit gene expression when the appropriate molecules (e.g., transcriptional activator proteins) are bound to the regulatory sequence(s).

By “operatively inserted” is meant that a nucleotide sequence of interest is positioned adjacent a nucleotide sequence that directs transcription and translation of the introduced nucleotide sequence of interest.

A “plant” refers to any plant or part of a plant at any stage of development. Therein are also included cuttings, cell or tissue cultures and seeds. As used in conjunction with the present invention, the term “plant tissue” includes, but is not limited to, whole plants, plant cells, plant organs, plant seeds, protoplasts, callus, cell cultures, and any groups of plant cells organized into structural and/or functional units.

By “plant cell” is meant structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

By “plant cell culture” is meant cultures of plant units such as, for example, protoplasts, cell culture cells, cells in plant tissues, pollen, pollen tubes, ovules, embryo sacs, zygotes and embryos at various stages of development.

By “plant material” is meant leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” refers to a group of plant cells organized into a structural and functional unit. Any tissue of a plant whether in a plant or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A “promoter” is an untranslated DNA sequence upstream of the coding region that contains the binding site for RNA polymerase II and initiates transcription of the DNA. The promoter region may also include other elements that act as regulators of gene expression.

A “protoplast” is an isolated plant cell without a cell wall or with only parts of the cell wall.

A “recombinant DNA molecule” is a combination of portions or parts of two or more DNA molecules that are joined together using recombinant DNA technology.

A “reduction of phenotypic expression” refers to the comparison of the phenotypic expression of the nucleic acid of interest to the eukaryotic cell in the presence of the RNA or chimeric genes of the invention, to the phenotypic expression of the nucleic acid of interest in the absence of the RNA or chimeric genes of the invention. The phenotypic expression in the presence of the chimeric RNA of the invention should thus be lower than the phenotypic expression in absence thereof. For example the expression would preferably be only about 25%, particularly only about 10%, more particularly only about 5% of the phenotypic expression in absence of the chimeric RNA. In one aspect of the invention the expression of the target gene should be completely inhibited for all practical purposes by the presence of the chimeric RNA or the chimeric gene encoding such an RNA.

“Reduction of phenotypic expression” of a nucleic acid where the phenotype is a qualitative trait means that in the presence of the chimeric RNA or gene of the invention, the phenotypic trait switches to a different discrete state when compared to a situation in which such RNA or gene is absent. A reduction of phenotypic expression of a nucleic acid may thus, be measured as a reduction in transcription of (part of) that nucleic acid, a reduction in translation of (part of) that nucleic acid, a reduction in the effect the presence of the transcribed RNA(s) or translated polypeptide(s) have on the eukaryotic cell or the organism, and will ultimately lead to altered phenotypic traits. It is clear that the reduction in phenotypic expression of a nucleic acid of interest, may be accompanied by or correlated to an increase in a phenotypic trait.

“Regulatory elements” refer to sequences involved in conferring the expression of a nucleotide sequence. Regulatory elements comprise a promoter operably linked to the nucleotide sequence of interest and termination signals. They also typically encompass sequences required for proper translation of the nucleotide sequence.

In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably the substantially similar nucleotide sequence encodes the polypeptide encoded by the reference nucleotide sequence. The percentage of identity between the substantially similar nucleotide sequence and the reference nucleotide sequence (number of complementary bases in the complementary sequence divided by total number of bases in the complementary sequence) desirably is at least 80%, more desirably 85%, preferably at least 90%, more preferably at least 95%, most preferably at least 99%.

A “selectable marker gene” is a gene whose expression in a plant cell gives the cell a selective advantage. The selective advantage possessed by the cells transformed with the selectable marker gene may be due to their ability to grow in the presence of a negative selective agent, such as an antibiotic or a herbicide, compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a plant cell gives the cell both, a negative and a positive selective advantage.

A “significant increase” refers to an increase in enzymatic activity that is larger than the margin of error inherent in the measurement technique, preferably an increase of about 2-fold or greater of the activity of the wild-type enzyme in the presence of the inhibitor, more preferably an increase of about 5-fold or greater, and most preferably an increase of about 10-fold or greater.

The terms “identical” or “percent identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The term “inhibit” when used in reference to activity (e.g. of an enzyme) refers to a partial or complete reduction in activity of the subject agent (e.g. enzyme).

The term “inhibitor” refers to a molecule or group of molecules that interferes with: (1) the expression, modification, regulation or activation of a nucleic acid sequence.

A “small interfering RNA” or “siRNA” is a double-stranded RNA molecule that is capable of inhibiting the expression of a gene with which it shares homology. In one embodiment the siRNA may be a “hairpin” or stem-loop RNA molecule, comprising a sense region, a loop region and an antisense region complementary to the sense region. In other embodiments the siRNA comprises two distinct RNA molecules that are non-covalently associated to form a duplex.

“Substantially identical,” in the context of two nucleic acid or protein sequences, refers to two or more sequences or subsequences that have at least 60%, preferably 80%, more preferably 90-95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff& Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, N.Y., which is hereby incorporated by reference. Generally, highly stringent hybridization and wash conditions are selected to be about 5.degree. C. lower than the thermal melting point (Tm) for the specific sequence at a defined ionic strength and pH. Typically, under “stringent conditions” a probe will hybridize to its target subsequence, but to no other sequences.

The T.sub.m is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on a filter in a Southern or northern blot is 50% formamide with 1 mg of heparin at 42.degree. C., with the hybridization being carried out overnight. An example of highly stringent wash conditions is 0.15M NaCl at 72.degree. C. for about 15 minutes. An example of stringent wash conditions is a 0.2.times.SSC wash at 65.degree. C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1.times.SSC at 45.degree. C. for 15 minutes. An example low stringency wash for a duplex of, e.g., more than 100 nucleotides, is 4-6.times.SSC at 40.degree. C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30.degree. C. Stringent conditions can also be achieved with the addition of destabilizing agents such as formamide. In general, a signal to noise ratio of 2.times.(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO.sub.4, 1 mM EDTA at 50.degree. C. with washing in 2.times.SSC, 0.1% SDS at 50.degree. C, more desirably in 7% sodium dodecyl sulfate (SDS), 0.5 M NaPO.sub.4, 1 mM EDTA at 50.degree. C with washing in 0.1.times.SSC, 0.1% SDS at 50.degree. C., more desirably still in 7% sodium dodecyl sulfate (SDS), 0.5 M NaPO.sub.4, 1 mM EDTA at 50.degree. C. with washing in 0.5.times.SSC, 0.1% SDS at 50.degree. C. preferably in 7% sodium dodecyl sulfate (SDS), 0.5 M NaPO.sub.4, 1 mM EDTA at 50.degree. C. with washing in 0.1.times.SSC, 0.1% SDS at 50.degree. C. more preferably in 7% sodium dodecyl sulfate (SDS), 0.5 M NaPO.sub.4, 1 mM EDTA at 50.degree. C. with washing in 0.1.times.SSC, 0.1% SDS at 65.degree. C.

A further indication that two nucleic acid sequences or proteins are substantially identical is that the protein encoded by the first nucleic acid is immunologically cross reactive with, or specifically binds to, the protein encoded by the second nucleic acid. Thus, the first protein is typically substantially identical to a second protein, for example, where the two proteins differ only by conservative substitutions.

Various biochemical and molecular biology methods are well known in the art. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements such as supplement 46 (April 1999), all of which are incorporated by reference herein.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a protein also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a protein is implicit in each described sequence.

Furthermore, one of skill will recognize that individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also, Creighton (1984) Proteins, W. H. Freeman and Company. In addition, individual substitutions, deletions or additions which alter, add or delete a single amino acid or a small percentage of amino acids in an encoded sequence are also “conservatively modified variations.”

The term “exogenous” when used with reference to a molecule (e.g., a nucleic acid) refers to a molecule that is not normally present in a cell, but can be introduced into a cell by one or more genetic, biochemical or other methods. By contrast, the term “endogenous” when used in reference to a molecule is one that is normally present in a particular cell at a particular developmental stage under particular environmental conditions.

“Modulation” refers to a change in the level or magnitude of an activity or process. The change can be either an increase or a decrease. For example, modulation of gene expression includes both gene activation and gene repression. Modulation can be assayed by determining any parameter that is indirectly or directly affected by the expression of the target gene. Such parameters include, e.g., changes in RNA or protein levels, changes in protein activity, changes in product levels, changes in downstream gene expression, changes in reporter gene transcription (luciferase, CAT,.beta.-galactosidase, .beta.-glucuronidase, green fluorescent protein (see, e.g., Mistili & Spector, Nature Biotechnology 15:961-964 (1997)); changes in signal transduction, phosphorylation and dephosphorylation, receptor-ligand interactions, second messenger concentrations (e.g., cGMP, cAMP, IP3, and Ca2+), and cell growth.

“Modulator” refers to an agent that alters (e.g. upregulates or down-regulates) expression or activity of a pathway and/or one or more components of a pathway.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The term “transformation” as used herein refers to both stable transformation and transient transformation.

A “subsequence” refers to a sequence of nucleic acids or amino acids that comprise a part of a longer sequence of nucleic acids or amino acids (e.g., protein) respectively.

“Target gene” generally means a polynucleotide comprising a region that encodes a polypeptide, or a polynucleotide region that regulates replication, transcription or translation or other processes important to expression of the polypeptide, or a polynucleotide comprising both a region that encodes a polypeptide and a region operably linked thereto that regulates expression.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277; Ishida Y. et al. (1996) Nature Biotech. 14:745-750) and particle-accelerated or “gene gun” transformation technology (Klein et al. (1987) Nature (London) 327:70-73; U.S. Pat. No. 4,945,050, all of which are incorporated herein by reference). Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990, all of which are incorporated herein by reference. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

The term “variants” when referring to, for example, polynucleotides encoding a polypeptide variant of a given reference polypeptide are polynucleotides that differ from the reference polypeptide but generally maintain their functional characteristics of the reference polypeptide. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

Generally, differences are limited so that the nucleotide sequences of the reference and variant are closely similar overall and, in many regions identical.

“Variants” of polynucleotides according to the present invention include, but are not limited to, nucleotide sequences which are at least 95% identical after alignment to the reference polynucleotide encoding the reference polypeptide. These variants can also have 96%, 97%, 98% and 99.999% sequence identity to the reference polynucleotide.

Nucleotide changes present in a variant polynucleotide may be silent, which means that these changes do not alter the amino acid sequences encoded by the reference polynucleotide.

### BRIEF DESCRIPTION OF THE FIGURES

FIG. 1 shows a diagram of the RNAi construct used to inhibit expression of the petunia EIN2 gene. A 1000 bp segment of the petunia EIN2 cDNA was cloned in antisense orientation, followed by a 450 bp segment of the EIN2 cDNA in sense orientation.

FIG. 2 (A-B) depicts pollinated wildtype (A) and transgenic petunia flowers (B). Wildtype (WT) flowers wilted 2 days after pollination (DAP), while flowers expressing an EIN2 RNAi construct remained turgid over 8 DAP.

FIG. 3 depicts PhEIN2 gene expression in flowers of EIN2 sense (sense 1-3) and EIN2 RNAi (RNAi 1-4) plants. PhEIN2 expression was approximately 3-fold lower in RNAi plants, indicating that RNAi was more effective than sense expression in inhibiting gene expression. Error bars represent standard error of the mean.

FIG. 4 depicts PhEIL1 expression in flowers of EIN2 sense, EIN2 RNAi, and wildtype flowers treated with 2 ppm ethylene for 16 hrs. PhEIL1 expression was strongly induced by exogenous ethylene in wildtype plants. There was a weak induction of PhEIL1 expression in EIN2 sense flowers and no significant induction in EIN2 RNAi flowers. Error bars represent standard error of the mean.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The information provided below is not admitted to be prior art to the present invention, but is provided solely to assist the understanding of the reader.

The details of one or more embodiments of the invention are set forth in the accompanying drawings and the description below. Other features, objects, and advantages of the invention will be apparent from the description and drawings, and from the claims.

The plant hormone ethylene regulates a wide range of developmental processes, including seed germination, abscission of leaves and flowers, stem elongation, and fruit ripening (Abeles et al., ETHYLENE IN PLANT BIOLOGY, San Diego, Academic Press, 1992). Significant progress has been made in identifying genes involved in both ethylene synthesis and perception. In plants, ethylene is synthesized from methionine and involves the conversion of S-adenosylmethionine to 1-aminocylopropane-1-carboxylic acid (ACC) by the enzyme ACC synthase. ACC is then directly converted to ethylene by ACC oxidase. (Mckeon and Yang, PLANT HORMONES AND THEIR ROLE IN PLANT GROWTH AND DEVELOPMENT, Boston, Martin Nijhoff,1987). Ethylene perception is controlled by a complex multicomponent pathway (Kieber, Annu Rev Plant Physiol Plant Mol Biol 48: 277-296, 1997). The first component of the pathway to be identified was an ethylene receptor gene from Arabidopsis, ETR1. This gene encodes a histidine kinase with homology to bacterial two-component systems (Chang et al., Science 284: 2148-2152, 1993). A total of five ethylene receptor genes have been cloned from Arabidopsis, ETR1, ETR2, ERS1, ERS2, and EIN4 (Hua et al., Science 269: 1712-1714, 1995; Hua et al., Plant Cell 10: 1321-1332, 1998; Sakai et al., PNAS 95: 5812-5817, 1998). Loss-of-function mutations in multiple ethylene receptors increase sensitivity to ethylene, indicating that the receptors are negative regulators of ethylene response (Hua and Meyerowitz, Cell 94: 261-271, 1998).

Downstream components of the pathway have also been identified in Arabidopsis. These include CTR1, which is homologous to the Raf family of serine/threonine kinases (Kieber et al., Cell 72: 427-441, 1993) and which interacts with the two ethylene receptor proteins ETR1 and ERS1 in a yeast two-hybrid system (Clark et al., PNAS 95: 5401-5406, 1998). EIN2 is an additional component of the ethylene signal transduction pathway. Epistasis analysis indicates that EIN2 acts downstream of CTR1. EIN2 is an integral membrane protein with 12 membrane spanning regions and is homologous to the Nramp metal-ion transport proteins (Alonso et al., Science 284: 2148-2152, 1999). EIN2 loss-of-function mutants are insensitive to ethylene, indicating that these proteins are positive regulators of ethylene response.

Several transcription factors that control the expression of ethylene-regulated genes have also been identified. The Arabidopsis EIN3 family contains several proteins that bind to an ethylene response element in the promoter of the ETHYLENE RESPONSE FACTOR 1 (ERF1) gene. ERF1 binds to the promoters of pathogenesis-related (PR) genes and regulates their expression (Solano et al., Genes Dev 12: 3703-3714, 1998). Loss-of-function mutations in EIN3 reduce sensitivity to ethylene, and overexpression of wild-type EIN3 cDNA results in constitutive ethylene response (Chao et al., Cell 89: 1133-1144, 1997). Therefore EIN3 is a positive regulator of ethylene response.

Isolation of several components of the ethylene synthesis and signal transduction pathways has made possible the manipulation of ethylene responses through genetic transformation. Tomato fruit ripening was altered through antisense expression of the ethylene biosynthesis genes ACC synthase (Oeller et al., Science 254: 437-439, 1991) and ACC oxidase (Hamilton et al., Nature 346: 284-287, 1990). Melon fruit ripening and abscission has also been delayed through antisense expression of an ACC oxidase gene (Ayub et al., Nature Biotech 14: 862-866, 1996). Similar results have been obtained by altering ethylene perception. For example, overexpression of etr1-1, a mutant form of an Arabidopsis ethylene receptor, decreased sensitivity to ethylene in tomato and petunia, delaying fruit ripening, flower senescence, and flower abscission (Wilkinson et al., Nature Biotech 15: 444-447, 1997). Overexpression of etr1-1 had no effect on ethylene production, indicating that these developmental responses were due to changes in ethylene perception. Downstream components of the ethylene signaling pathway have also been used to manipulate ethylene response. For example antisense expression of the tomato EIN3 genes LeEIL1, LeEIL2, and LeEIL3 greatly reduced ethylene sensitivity throughout the plant. Like etr1-1 overexpression, antisense expression of the LeEIL genes did not cause changes in ethylene synthesis (Tieman et al., Plant J. 26: 47-58, 2001).

Manipulation of ethylene-related genes can be applied to several different crops to regulate agronomically important traits. One of the most promising commercial applications of this technology is in the prevention of flower senescence and abscission. Senescence is the ageing of a plant tissue and is often associated with chlorophyll and protein degradation and loss of turgor. Abscission is the separation of a plant part resulting from the degradation of specialized cells which comprise an abscission zone.

Although the methods described above have been used to regulate flower aging, there are several disadvantages to these approaches for commercial applications. The greatest disadvantage is that in all cases constitutive promoters were used to drive transgene expression, resulting in changes in ethylene response throughout the plant. Although ethylene insensitivity may be useful in flowers to prevent senescence, it has been shown to have negative effects in other tissues such as roots and stems. For example, overexpression of the etr1-1 mutant receptor in tobacco increased susceptibility to fungal pathogens infecting roots (Knoester et al., PNAS 95: 1933-1937, 1998). Furthermore, the ethylene-insensitive Neverripe tomato mutant (that contains a dominant mutation in an ethylene receptor gene) displays reduced adventitious rooting (Clark et al., Plant Physiol. 121: 53-59, 1999). Therefore altering ethylene sensitivity through constitutive changes in ethylene receptor gene expression is likely to cause negative effects in petunia as well.

It would be beneficial then to limit changes in ethylene response to the targeted tissue, for example in the flower portion of the plant. However, since ethylene is readily diffusible throughout the plant, it would be difficult to reduce ethylene levels in any one tissue simply by reducing synthesis in a specific area. Therefore, it would be difficult to achieve tissue-specific changes in ethylene response through manipulation of the ethylene biosynthesis genes. Furthermore, ACC synthase and ACC oxidase are encoded by multigene families in several species, making it necessary to clone and characterize several different family members before the gene (or genes) that regulate ethylene synthesis in a specific tissue can be identified. Therefore, manipulation of ethylene perception, as opposed to ethylene production, would be a more effective way to engineer tissue specific changes in ethylene response.

For example, we have developed a novel method for engineering ethylene insensitivity in petunia through manipulation of the petunia EIN2 (PhEIN2) gene. EIN2 is a transmembrane protein that acts downstream of the ethylene receptors and is encoded by a single gene in Arabidopsis (Alonso et al., Science 284: 2148-2152, 1999). Our work show that cosuppression of PhEIN2 through sense expression of a 1.1 kb segment of this gene is more effective than antisense expression in delaying flower senescence (WO03034814). Here we have discovered that use of an RNAi construct is even more effective than sense expression in reducing PhEIN2 expression in petunia, resulting in stronger ethylene insensitivity, greater flower longevity, and a higher percentage of transgenic lines with delayed flower senescence.

Those skilled in the art would recognize that no technique, prior to the invention described herein, allows for the generation of transgenic plants having a specific ethylene signaling gene to be down regulated through double stranded RNA technology. The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al, International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

Double stranded RNA (dsRNA) useful in accordance with the invention is derived from an “exogenous or endogenous template”. Preferably, the template is of “endogenous template”. Such a template may be all or part of a nucleotide sequence endogenous to the plant; it may be a DNA gene sequence or a cDNA produced from an mRNA isolated from the plant, for example by reverse transcriptase. When the template is all or a part of a DNA gene sequence, it is preferred if it is from one or more or all exons of the gene. Additionally, all or part of an ethylene signaling gene may form an endogenous template, if it is expressed in the plant in such a way that the ethylene regulates its response in a plant or a plant cell, e.g. expression from EIN2 integrated into the host cell chromosome. While the dsRNA is derived from an endogenous or exogenous template, there is no limitation on the manner in which it could be synthesized. In recent review article in Ambion (TechNotes (ambion). 2003. Volume: 10, Number: 3.), it was reported that currently there are five methods for generating siRNAs for gene silencing studies:
1. Chemical synthesis
2. In vitro transcription
3. Digestion of long dsRNA by an RNase III family enzyme (e.g. Dicer, RNase III)
4. Expression in cells from an siRNA expression plasmid or viral vector
5. Expression in cells from a PCR-derived siRNA expression cassette

It was reported that the first three methods involve in vitro preparation of siRNA that is then introduced directly into mammalian cells by transfection, electroporation, or by another method. The last two methods depend on the transfection of DNA-based vectors and cassettes that express siRNAs within the cells. It was further reported, in the same article, that each of these methods has its advantages and drawbacks and the best method for generating siRNAs will depend on the goals of the experiment. Thus, RNAi may be synthesized in vitro or in vivo, using manual and/or automated procedures. In vitro synthesis may be chemical or enzymatic, for example using cloned RNA polymerase (e.g., T3, T7, SP6) for transcription of the endogenous DNA (or cDNA) template, or a mixture of both.

In vivo, the dsRNA may be synthesised using recombinant techniques well known in the art (see e.g., Sambrook, et al., MOLECULAR CLONING; A LABORATORY MANUAL, SECOND EDITION (1989); DNA CLONING, VOLUMES I AND II (D. N Glover ed. 1985); OLIGONUCLEOTIDE SYNTHESIS (M. J. Gait ed, 1984); NUCLEIC ACID HYBRIDISATION (B. D. Hames & S. J. Higgins eds. 1984); TRANSCRIPTION AND TRANSLATION (B. D. Hames & S. J. Higgins eds. 1984); ANIMAL CELL CULTURE (R. I. Freshney ed. 1986); IMMOBILISED CELLS AND ENZYMES (IRL Press, 1986); B. Perbal, A PRACTICAL GUIDE TO MOLECULAR CLONING (1984); the series, METHODS IN ENZYMOLOGY (Academic Press, Inc.); GENE TRANSFER VECTORS FOR MAMMALIAN CELLS (J. H. Miller and M. P. Calos eds. 1987, Cold Spring Harbor Laboratory), Methods in Enzymology Vol. 154 and Vol. 155 (Wu and Grossman, and Wu, eds., respectively), Mayer and Walker, eds. (1987), IMMUNOCHEMICAL METHODS IN CELL AND MOLECULAR BIOLOGY (Academic Press, London), Scopes, (1987), PROTEIN PURIFICATION: PRINCIPLES AND PRACTICE, Second Edition (Springer-Verlag, N.Y.), and HANDBOOK OF EXPERIMENTAL IMMUNOLOGY, VOLUMES I-IV (D. M. Weir and C. C. Blackwell eds 1986). A very recent review article on ways for making siRNA was published by in TechNotes (ambion). 2003. Volume: 10, Number: 3.

Thus, bacterial cells can be transformed with an expression vector which comprises the DNA template from which the dsRNA is to be derived. Alternatively, the cells, of a plant for example, in which inhibition of gene expression is required may be transformed with an expression vector or by other means. Bidirectional transcription of one or more copies of the template may be by endogenous RNA polymerase of the transformed cell or by a cloned RNA polymerase (e.g., T3, T7, SP6) coded for by the expression vector or a different expression vector. The use and production of an expression construct are known in the art (see WO98/32016; U.S. Pat. Nos. 5,593,874, 5,698,425, 5712,135, 5,789,214, and 5,804,693). Inhibition of gene expression may be targeted by specific transcription in an organ, tissue, or cell type; an environmental condition (e.g. temperature, chemical); and/or engineering transcription at a developmental stage or age, especially when the dsRNA is synthesised in vivo in the plant cell for example. dsRNA may also be delivered to specific tissues or cell types using known gene delivery systems. Components of these systems include the seed-specific lectin promoter (Wesley et al., Plant J 27: 581-590, 2001) and the flower specific promoter from APETALA3 (Jack et al, Cell 68: 683-697, 1992). These vectors are listed solely by way of illustration of the many commercially available and well known vectors that are available to those of skill in the art.

If synthesized outside the cell, the RNA may be purified prior to introduction into the cell. Purification may be by extraction with a solvent (such as phenol/chloroform) or resin, precipitation (for example in ethanol), electrophoresis, chromatography, or a combination thereof. However, purification may result in loss of dsRNA and may therefore be minimal or not carried out at all. The RNA may be dried for storage or dissolved in an aqueous solution, which may contain buffers or salts to promote annealing, and/or stabilization of the RNA strands.

dsRNA useful in the present invention includes dsRNA which contains one or more modified bases, and dsRNA with a backbone modified for stability or for other reasons. For example, the phosphodiester linkages of natural RNA may be modified to include at least one of a nitrogen or sulfur heteroatom. Moreover, dsRNA comprising unusual bases, such as inosine, or modified bases, such as tritylated bases, to name just two examples, can be used in the invention. It will be appreciated that a great variety of modifications have been made to RNA that serve many useful purposes known to those of skill in the art. The term dsRNA as it is employed herein embraces such chemically, enzymatically or metabolically modified forms of dsRNA, provided that it is derived from an endogenous template.

The double-stranded structure may be formed by a single self-complementary RNA strand or two separate complementary RNA strands. RNA duplex formation may be initiated either inside or outside the plant cell.

The dsRNA comprises a double stranded structure, the sequence of which is “substantially identical” to at least a part of the target gene. “Identity”, as known in the art, is the relationship between two or more polynucleotide (or polypeptide) sequences, as determined by comparing the sequences. In the art, identity also means the degree of sequence relatedness between polynucleotide sequences, as determined by the match between strings of such sequences. Identity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide sequences, the term is well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods commonly employed to determine identity between sequences include, but are not limited to those disclosed in Carillo, H., and Lipman, D., SIAM J. Applied Math., 48:1073 (1988). Preferred methods to determine identity are designed to give the largest match between the sequences tested. Methods to determine identity are codified in computer programs. Computer program methods to determine identity between two sequences include, but are not limited to, GCG program package (Devereux, J., et al., Nucleic Acids Research 12(1): 387 (1984)), BLASTP, BLASTN, and FASTA (Atschul, S. F. et al., J. Molec. Biol. 215: 403 (1990)). Another software package well known in the art for carrying out this procedure is the CLUSTAL program. It compares the sequences of two polynucleotides and finds the optimal alignment by inserting spaces in either sequence as appropriate. The identity for an optimal alignment can also be calculated using a software package such as BLASTx. This program aligns the largest stretch of similar sequence and assigns a value to the fit. For any one pattern comparison several regions of similarity may be found, each having a different score. One skilled in the art will appreciate that two polynucleotides of different lengths may be compared over the entire length of the longer fragment. Alternatively small regions may be compared. Normally sequences of the same length are compared for a useful comparison to be made.

It is preferred to have 100% sequence identity between the inhibitory RNA and the part of the target gene. However, dsRNA having 70%, 80% or greater than 90% or 95% sequence identity may be used in the present invention with good results. Thus sequence variations that might be expected due to genetic mutation, strain polymorphism, or evolutionary divergence can be tolerated.

The duplex region of the RNA may have a nucleotide sequence that is capable of hybridizing with a portion of the target gene transcript (e.g., 400 mM NaCl, 40 mM PIPES pH 6.4, 1 mM EDTA, 50.degree. C. or 70.degree. C. hybridization for 12-16 hours; followed by washing).

While the optimum length of the dsRNA may vary according to the target gene and experimental conditions, the duplex region of the RNA may be at least 25, 50, 100, 200, 300, 400 or more bases long.

Target genes may be cellular genes present in the genome. It is preferred if the dsRNA is substantially identical to the whole of the target gene, i.e. the coding portion of the gene. However, the dsRNA can be substantially identical to a part of the target gene. The size of this part depends on the particular target gene and can be determined by those skilled in the art by varying the size of the dsRNA and observing whether expression of the gene has been inhibited.

In the present invention, one application would be the use of dsRNA to inhibit a target gene which affects or is likely to affects flower development, i.e. it can be used to lengthen flower longevity.

In the increasing of flower longevity, the target gene may be one which is required for initiation or maintenance of the flower, or which has been identified as being associated with flower development.

In controlling flower longevity, the dsRNA can be brought into contact with the cells or tissue in any of the flower parts.

The following classes of possible target genes are examples, but not limited to, of the genes which the present invention may used to inhibit: APETALA3, AGAMOUS, and PISTILLA TA.

Introducing into the cell an RNA comprising a double stranded structure having a nucleotide sequence which is substantially identical to at least a part of the target gene and which is derived from an exogenous or an endogenous template.

Inhibition of the expression of a target gene can be verified by observing or detecting an absence or observable decrease in the level of protein encoded by a target gene (this may be detected by for example a specific antibody or other techniques known to the skilled person) and/or mRNA product from a target gene (this may be detected by for example hybridization studies) and/or phenotype associated with expression of the gene.

The amount of dsRNA administered to an object (e.g., cells of a flower) for effective gene inhibition will vary according to a variety of factors, including the route of application, the age, size and condition of the plant, the gene which is to be inhibited, the plant species to be treated and so on. Similarly, in mammals, those skilled in the art found that, when injecting 10 μl into cell of the early embryo, solutions having dsRNA at a concentration in the range of from 0.01 to 40 mg/ml, preferably 0.1 to 4 mg/ml and most preferable 0.1 to 2 mg/ml are effective.

RNA may be introduced into the cell intracellularly or extracellularly. Physical methods of introducing nucleic acids may also be used in this respect. The dsRNA may be administered using the microinjection techniques described in Zernicka-Goetz,. et al. Development 124, 1133-1137 (1997) and Wianny, et al. Chromosoma 107, 430-439 (1998). Other physical methods of introducing nucleic acids intracellularly include bombardment by particles covered by the RNA, for example gene gun technology in which the dsRNA is immobilized on gold particles and fired directly at the site of wounding. Thus, the invention provides the use of an RNA comprising a double stranded structure having a nucleotide sequence, which is substantially identical to at least a part of a target gene in a cell and which is derived from an exogenous or an endogenous template, in a gene gun for inhibiting the expression of the target gene. Further, there is provided a composition suitable for gene gun comprising: an RNA comprising a double stranded structure having a nucleotide sequence which is substantially identical to at least a part of a target gene in a cell and which is derived from an exogenous or an endogenous template; and gold particles. An alternative physical method includes electroporation of cell membranes in the presence of the RNA. dsRNA can be introduced into cells by electoporation using conditions similar to those generally applied to cultured cells. Precise conditions for electroporation depend on the device used to produce the electro-shock and the dimensions of the chamber used to hold the cells. Any known gene delivery technique can be used to administer the RNA. Other methods known in the art for introducing nucleic acids to cells may be used, such as lipid-mediated carrier transport, chemical-mediated transport, such as calcium phosphate, and the like. Thus, the RNA may be introduced along with components that perform one or more of the following activities: enhance RNA uptake by the cell, promote annealing of the duplex strands, stabilize the annealed strands, or otherwise increase inhibition of the target gene. A transgenic plant that expresses RNA from a recombinant construct may be produced by introducing the construct into a plant cell (e.g., embryogenic cell, non-embryogenic cell . . . ).

RNA fragments are introduced in the plants cell by different transformation methods. For example, the RNA fragments are transferred to the host cells using particle bombardment. In another preferred embodiment, the RNA fragments are introduced into the protoplasts or other types of cells by PEG-mediated transformation as described in Lebel et al. (1995) Theor. Appl. Genet. 91: 899-906 or by electroporation. In another preferred embodiment, other techniques, such as microinjection of the RNA fragments, are used.

Nucleic acid molecules of the present invention are transformed into plant cells using methods well-known in the art or described below. For example, microprojectile bombardment, microinjection or Agrobacterium-mediated transformation is used. Also, transformation of protoplasts with DNA molecules of the present invention using electroporation or chemical treatment (e.g. PEG treatment) is used.

Alternatively, viral vectors are used to introduce RNA fragments of the present invention into plant cells, e.g. through so-called agroinfection. In another preferred embodiment, RNA fragments of the present invention into plant cells are introduced into plant cells by vacuum infiltration or by rubbing on leaf surface.

Plant Transformation Technology

DNA molecules and RNA molecules of the present invention are incorporated in plant or bacterial cells using conventional recombinant DNA technology. Generally, a DNA or an RNA molecule of the present invention is comprised in a transformation vector. A large number of such vector systems known in the art may be used, such as plasmids. The components of the expression system are also modified, e.g. to increase expression of the introduced RNA fragments. For example, truncated sequences, nucleotide substitutions or other modifications may be employed. Expression systems known in the art may be used to transform virtually any crop plant cell under suitable conditions. A transgene comprising a DNA molecule of the present invention is preferably stably transformed and integrated into the genome of the host cells. Transformed cells are preferably regenerated into whole plants. Detailed description of transformation techniques are within the knowledge of those skilled in the art (e.g., US20030024014).

Reporter genes or selectable marker genes may be included in the expression cassette. Examples of suitable reporter genes known in the art can be found in, for example, Jefferson et al. (1991) in Plant Molecular Biology Manual, ed. Gelvin et al. (Kluwer Academic Publishers), pp. 1-33; DeWet et al. (1987) Mol. Cell. Biol. 7:725-737; Goff et al. (1990) EMBO J. 9:2517-2522; Kain et al. (1995) Bio Techniques 19:650-655; and Chiu et al. (1996) Current Biology 6:325-330.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella et al. (1983) EMBO J. 2:987-992); methotrexate (Herrera Estrella et al. (1983) Nature 303:209-213; Meijer et al. (1991) Plant Mol. Biol. 16:807-820); hygromycin (Waldron et al. (1985) Plant Mol. Biol. 5:103-108; Zhijian et al. (1995) Plant Science 108:219-227); streptomycin (Jones et al. (1987) Mol. Gen. Genet 210:86-91); spectinomycin (Bretagne-Sagnard et al. (1996) Transgenic Res. 5:131-137); bleomycin (Hille et al. (1990) Plant Mol. Biol 7:171-176); sulfonamide (Guerineau et al. 1990) Plant Mol. Biol. 15:127-136); bromoxynil (Stalker et al. (1988) Science 242:41 9423); glyphosate (Shaw et al. (1986) Science 233:478481); phosphinothricin (DeBlock et al. (1987) EMBO J. 6:2513-2518).

Other genes that could serve utility in the recovery of transgenic events but might not be required in the final product would include, but are not limited to, examples such as GUS (b-glucoronidase; Jefferson (1987) Plant Mol. Biol. Rep. 5:387), GFP (green florescence protein; Chalfie et al. (1994) Science 263:802), luciferase (Riggs et al. (1987) Nucleic Acids Res. 15(19):8115 and Luehrsen et al. (1992) Methods Enzymol. 216:397-414) and the maize genes encoding for anthocyanin production (Ludwig et al. (1990) Science 247:449).

The expression cassette comprising a promoter sequence operably linked to a heterologous nucleotide sequence of interest can be used to transform any plant. In this manner, genetically modified plants, plant cells, plant tissue, seed, and the like can be obtained.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Dafta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional techniques. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize 1n2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Constitutive promoters include, for example, the core promoter of the Rsyn7 promoter and other constitutive promoters disclosed in WO 99/43838 and U.S. Pat. No. 6,072,050; the core CAMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142.

Where low level expression is desired, weak promoters may be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142.

“Tissue-preferred” promoters can be used to target a gene expression within a particular tissue. Tissue-preferred promoters include Yamamoto et al. (1997) Plant J. 12(2)255-265; Kawamata et al. (1997) Plant Cell Physiol. 38(7):792-803; Hansen et al (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase); and ce1A (cellulose synthase). Gama-zein is a preferred endosperm-specific promoter. Glob-1 is a preferred embryo-specific promoter. For dicots, seed-specific promoters include, but are not limited to, bean.beta.-phaseolin, napin,.beta.-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc.

Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and may be selected from the many available from the literature or isolated de novo from various compatible species. See, for example, Hire et al. (1992) Plant Mol. Biol. 20(2): 207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):l 1′-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a beta.-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed ro1C and ro1D root-inducing genes of Agrobacterium rhizogenes (see Plant Science (Limerick) 79(1):69-76). They concluded that enhancer and tissue-specific DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to npt11 (neomycin phosphotransferase 11) has shown similar characteristics. Additional root-preferred promoters include the VfENOD-GRP3 gene promoter (Kuster et al. (1995) Plant Mol. Biol. 29(4):759-772); and ro1B promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation may be accomplished by transactivation of a silent plastid-bome transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

Plants transformed in accordance with the present invention may be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, flowers, cut flowers (e.g., roses, carnation . . . ), and woody plants such as coniferous and deciduous trees. Once a desired nucleotide sequence has been transformed into a particular plant species, it may be propagated in that species or moved into other varieties of the same species, particularly including commercial varieties, using traditional breeding techniques.

Requirements for Construction of Plant Expression Cassettes

Nucleic acid sequences intended for expression in transgenic plants are first assembled in expression cassettes behind a suitable promoter expressible in plants. The expression cassettes may also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described infra. The following is a description of various components of typical expression cassettes.

1. Promoters

The selection of the promoter used in expression cassettes determine the spatial and temporal expression pattern of the transgene in the transgenic plant. Selected promoters express transgenes in specific cell types (such as leaf epidermal cells, mesophyll cells, root cortex cells) or in specific tissues or organs (roots, leaves or flowers, for example) and the selection reflects the desired location of accumulation of the gene product. Alternatively, the selected promoter drives expression of the gene under various inducing conditions.

Promoters vary in their strength, i.e., ability to promote transcription. Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044).

A preferred category of promoters is that which is wound inducible. Numerous promoters have been described which are expressed at wound sites. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Preferred tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A preferred promoter is the maize PEPC promoter from the phosphoenol carboxylase gene (Hudspeth & Grula, Plant Molec.Biol. 12: 579-589 (1989)). A preferred promoter for root specific expression is that described by de Framond (FEBS 290: 103-106 (1991); EP 0 452 269 and a further preferred rootspecific promoter is that from the T-1 gene provided by this invention. A preferred stem specific promoter is that described in U.S. Pat. No. 5,625,136 and which drives expression of the maize trpA gene.

Preferred embodiments of the invention are transgenic plants expressing nucleotide sequence in a flower-specific fashion. Promoters suitable for expression in floral tissue include, but not limited to, APETALA3 (Jack et al., Cell 68: 683-697, 1992) and FLORAL BINDING PROTEIN 1 (FBP1) (Angenent et al., Plant J 4: 101-112, 1993).

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

3. Sequences for the Enhancement or Regulation of Expression

Numerous sequences have been found to enhance gene expression from within the transcriptional unit and these sequences can be used in conjunction with the genes of this invention to increase their expression in transgenic plants. For example, various intron sequences such as introns of the maize Adh1 gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene may be genetically engineered by altering the coding sequence for optimal expression in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al, Bio/technol. 11: 194 (1993)).

In another preferred embodiment, an RNA molecule of the present invention is directly transformed into the plastid genome. Plastid transformation technology is extensively described in U.S. Pat. Nos. 5,451,513,5,545,817, and 5,545,818, in PCT application no. WO 95/16783, and in McBride et al. (1994) Proc. Natl. Acad. Sci. USA 91,7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87,8526-8530; Staub, J. M., and Maliga, P. (1992) Plant Cell 4,39-45). The presence of cloning sites between these markers allowed creation of a plastid targeting vector for introduction of foreign DNA molecules (Staub, J. M., and Maliga, P. (1993) EMBO J. 12,601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90,913-917). Previously, this marker had been used successfully for high-frequency transformation of the plastid genome of the green alga Chlamydomonas reinhardtii (Goldschmidt-Clermont, M. (1991) Nucl. Acids Res. 19: 4083-4089). Other selectable markers useful for plastid transformation are known in the art and are encompassed within the scope of the invention.

Construction of Plant Transformation Vectors

Numerous transformation vectors available for plant transformation are known to those of ordinary skill in the plant transformation arts, and the genes pertinent to this invention can be used in conjunction with any such vectors. The selection of vector depends upon the preferred transformation technique and the target species for transformation. For certain target species, different antibiotic or herbicide selection markers are preferred. Selection markers used routinely in transformation include the npt11 gene, which confers resistance to kanamycin and related antibiotics (Messing & Vierra. Gene 19: 259-268 (1982); Bevan et al., Nature 304: 184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), the manA gene, which allows for positive selection in the presence of mannose (Miles and Guest (1984) Gene, 32: 41-48; U.S. Patent No. 5,767,378), and the dhfr gene, which confers resistance to methotrexate (Bourouis et al., EMBO J. 2 (7): 1099-1104 (1983)), and the EPSPS gene, which confers resistance to glyphosate (U.S. Pat. Nos. 4,940,935 and 5,188,642).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984). Typical vectors suitable for Agrobacterium transformation include the binary vectors pCIB200 and pCIB2001, as well as the binary vector pCIB 10 and hygromycin selection derivatives thereof. (See, for example, U.S. Pat. No. 5,639,949).

2. Vectors Suitable for Non-Agrobacterium Transformation

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Typical vectors suitable for non-Agrobacterium transformation include pCIB3064, pSOG 19, and pSOG35. (See, for example, U.S. Pat. No. 5,639,949).

C. Transformation Techniques

Once the DNA sequence of interest is cloned into an expression system, it is transformed into a plant cell. Methods for transformation and regeneration of plants are well known in the art. For example, Ti plasmid vectors have been utilized for the delivery of foreign DNA, as well as direct DNA uptake, liposomes, electroporation, micro-injection, and microprojectiles. In addition, bacteria from the genus Agrobacterium can be utilized to transform plant cells.

Transformation techniques for dicotyledons are well known in the art and include Agrobacterium-based techniques and techniques that do not require Agrobacterium. Non Agrobacterium techniques involve the uptake of exogenous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art. The methods can result in plant cells comprising the RNA fragments of the present invention, wherein the expression of said target gene in said plant cell is altered by said RNA fragments, a plant and the progeny thereof derived from the plant cell, and seeds derived from the plant.

The invention also provides an RNA comprising a double stranded structure having a nucleotide sequence which is substantially identical to at least a part of a target gene in a plant cell for example and which is derived from an exogenous or endogenous template for use in Agriculture wherein the target gene is modulated by ethylene.

The present invention may be used alone or as a component of a kit having at least one of the reagents necessary to carry out the in vitro or in vivo introduction of RNA to subjects. Preferred components are the dsRNA and a vehicle that promotes introduction of the dsRNA. Such a kit may also include instructions to allow a user of the kit to practice the invention.

According to a further aspect of the present invention, there is provided a method for inhibiting the expression of a target gene in a plant cell for example, the method comprising:
introducing into the cell an RNA comprising a double stranded structure having a nucleotide sequence which is substantially identical to at least a part of the target gene; and optionally verifying inhibition of expression of the target gene. In this aspect, it is preferred that the RNA is derived from an exogenous or an endogenous template; wherein the target gene is modulated by ethylene.

In a further aspect, the present invention provides a method for treating or preventing a condition such as flower abscission, caused by a target gene in a plant, comprising: bringing the target gene into contact with dsRNA having a sequence which is substantially identical to at least a part of the target gene; wherein the target gene is modulated by ethylene. In this aspect, it is preferred that the RNA is derived from an endogenous template.

The invention also provides a plant cell, for example, containing an expression construct, the construct coding for an RNA which forms a double stranded structure having a nucleotide sequence which is substantially identical to at least a part of a target gene and which is derived from an endogenous template, as well as a transgenic plant containing such a cell.

The present invention relates to methods for regulating gene expression in plant cells.

The present invention utilizes an RNA fragment substantially identical to at least a fragment of a target gene to alter the expression of the gene in a plant cell. In a first embodiment, the invention provides a method for altering expression of a target gene in a plant cell comprising introducing into a plant cell an RNA fragments substantially identical to at least a fragment of a target gene, wherein said RNA are capable of forming a double-stranded RNA molecule, wherein the expression of said target gene in said cell is altered.

In another preferred embodiment, the RNA fragments are comprised in two different RNA molecules. In this case, the RNA fragments are mixed before being introduced into said cell, e.g. under conditions allowing them to form a double-stranded RNA molecule. In another preferred embodiment, the RNA fragments are introduced into said cell sequentially. Preferably, the time interval between the introduction of each of the RNA molecules is short, preferably less than one hour.

In yet another embodiment, the RNA fragments are comprised in one RNA molecule. By using one single RNA molecule, the two complementary RNA fragments are in close proximity such that pairing and double strand formation is favored. In such case, the RNA molecule is preferably capable of folding such that said RNA fragments comprised therein form a double-stranded region. In this case, the complementary parts of the RNA fragments recognize one another, pair with each other and form the double-stranded RNA molecule. In a preferred embodiment, the RNA fragments are incubated under conditions allowing them to form a double-stranded RNA molecule prior to introduction into the cell. In yet another embodiment, the RNA molecule comprises a linker between the sense RNA fragment and the antisense RNA fragment. The linker preferably comprises a RNA sequence encoded by an expression cassette comprising a functional gene, e.g. a selectable marker gene. In another embodiment, the linker comprises a RNA sequence encoded by regulatory sequences, which e.g. comprise intron processing signals.

In yet another preferred embodiment, the dsRNA construct further comprises a promoter operably linked to said dsRNA and might further comprise said dsRNA molecule. In another embodiment the promoter is a heterologous promoter, for example a tissue specific promoter, a developmentally regulated promoter, a constitutive promoter, divergent or an inducible promoter. In another preferred embodiment the gene is an heterologous gene in said plant cell. Termination signal are also optionally included in the DNA molecules.

The single RNA molecule or the two distinct RNA molecules are preferably capable of forming a double-stranded region, in which the complementary parts of the RNA fragments recognize one another, pair with each other and form the double-stranded RNA molecule.

The following are only a few examples where our invention will likely find application. It should be understood that our invention is not limited to these examples. Other important applications of our invention would be readily recognized by those of ordinary skills in the art. Other uses which are potentially recognizable by those of ordinary skills in the art are also part of our invention.

The prior art documents mentioned herein are incorporated to the fullest extent permitted by law.

### EXAMPLES

The present invention will now be described further in the following examples. Reference is made to the accompanying drawings:

Production of Transgenic Petunia Plants

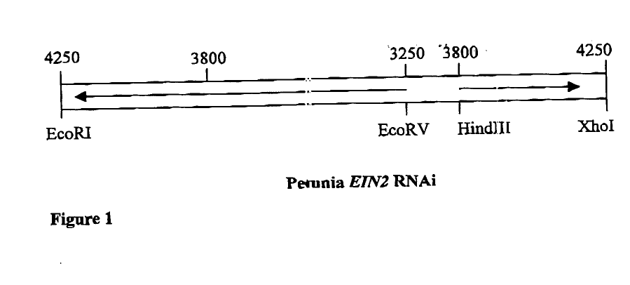

A 1.1 kb segment of the PhEIN2 cDNA spanning from nucleotide 2824 to 3940 was cloned into a vector downstream from a cauliflower mosaic virus promoter (CAMV 35S) and upstream of the Agrobacterium nopaline synthase (nos) terminus region. Two separate constructs were made with the PhEIN2 cDNA fragment in either the sense or antisense orientation. For the RNAi construct, a 1.0 kb EcORI/EcORV restriction fragment from the PhEIN2 cDNA was cloned into the EcORIand EcORV sites of pBluescript SK+(Stratagene). This fragment spanned from bases 3250 to 4250 of the petunia cDNA. A 450 bp fragment which spanned from bases 3800 to 4250 of the PhEIN2 cDNA was then amplified by PCR, adding a HindIII site to the 5′ end and BamHI and XhoI sites to the 3′ end. This PCR product was then cloned into the HindIII and XhoI sites of pBluescript at the 5′ end of the 1.0 kb restriction fragment (FIG. 1). The RNAi construct was excised from pBluescript as a BamHI fragment and cloned downstream of the constitutive CAMV35S promoter and upstream of the nos terminus region. This entire construct was then excised as a NotI fragment and cloned into the NotI site of a binary transformation vector.

The transformation vectors were transferred to Agrobacterium through triparental mating. A group of petunia plants (cv Mitchell Diploid) were transformed with this construct through Agrobacterium-mediated transformation (Horsch et al., Science 227: 1229-1231, 1985).

All plants were grown under standard greenhouse conditions. Presence of the transgene was confirmed in To plants through PCR by amplifying a segment of the kanamycin resistance gene. Plants were then screened for alterations in ethylene-sensitivity by two methods. In the first, flowers were cut from the plant on the day before anthesis and placed in vials of water. The flowers were then sealed in a glass container and treated with 2-5 ppm ethylene for 16 hrs. The flowers were then placed in a growth room and the day on which the flowers completely wilted was recorded. In the second assays, flowers were pollinated on the plant on the day before anthesis and the number of days to wilting was recorded. Seed was collected from plants that displayed increased flower longevity and these plants were also analyzed for presence of the transgene and flower longevity in subsequent generations.

For quantification of PhEIL1 and PhEIN2 gene expression, detached flowers were treated in a sealed glass container with ethylene concentrations ranging from 0.01 to 10 ppm. Flowers were treated for 4 hrs. Total RNAs were extracted from open flowers using an RNeasy Plant Mini Kit (Qiagen, Valencia, Calif.) and treated with RNase-Free DNase Set (Qiagen, Valencia, Calif.). Real-time quantitative PCR was performed on 250 ng total RNA in a 25 μl reaction volume using TaqMan Reverse Transcription Reagents and SYBR Green PCR Core Reagents (Applied Biosystems, Foster City, Calif.) on an Applied Biosystems GeneAmp 5700 sequence-detection system. The primers designed with primer express software (Applied Biosystems) for each gene were: PhEIN2 forward primer, TGTGTTTTTCTGGCTGCAACTG; PhEIN2 reverse primer, GCACTGCCATTGTCCAAGAGA; PhEIL1 forward primer, GCCTTTTCCATCTCCACTTACTATTG; PhEIL1 reverse primer, TGGATATCAAGCCCCAAATTAAA. The final concentration of each primer was 300 nM. RT-PCR conditions were as follows: 48° C. for 30 min, 95° C. for 10 min followed by 40 cycles of 95° C. for 15 sec and 60° C. for 1 min. Sense-strand RNA, synthesized as described in Tieman et al., Plant J 26: 47-58, (2001), was used as a standard to determine absolute levels of PhEIN2 and PhEIL1 mRNA.

We previously demonstrated that cosuppression of the petunia EIN2 (PhEIN2) gene through overexpression of a 1.1 kb fragment in sense orientation delays flower senescence (WO03034814). Studies with several plant species have shown that use of a double-stranded RNA (RNAi) construct is more effective than overexpression for cosuppression of native genes. However, RNAi has not been evaluated in petunia.

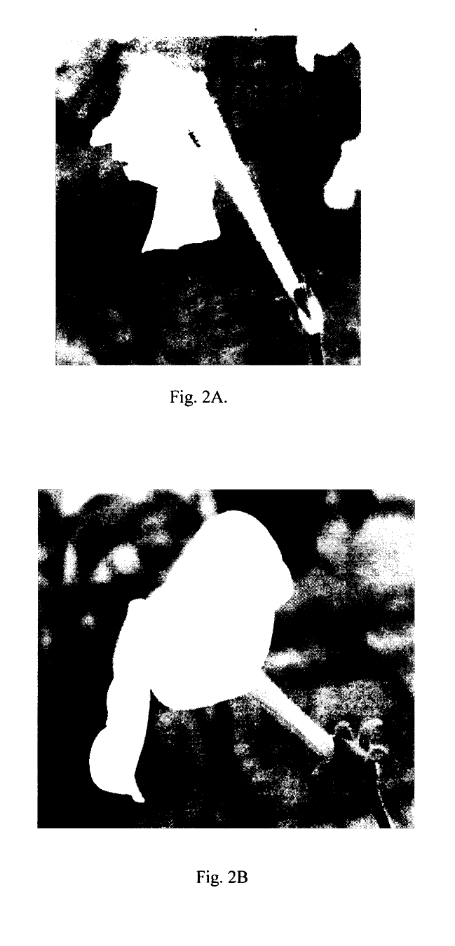

In this study, we compared the effectiveness of sense expression and RNAi in delaying petunia flower senescence through cosuppression of PhEIN2. Over 70 independent transgenic petunia lines were generated for each construct and the primary transformants were evaluated for changes in flower longevity. Wildtype flowers last an average of 2 days after pollination (DAP), while flowers from 9% of the EIN2 RNAi lines remained turgid more than 20 DAP (Table 1 and FIG. 2). Table 1: lists the percentage of primary transformant petunia plants exhibiting ethylene-insensitive phenotypes. Approximately 100 independent transgenic lines were evaluated for the EIN2 sense construct and 70 lines for the EIN2 RNAi construct. Thirty-four percent of the RNAi lines showed a significant decrease in ethylene sensitivity, compared to 8 percent for the EIN2 sense lines.

| Flower longevity (DAP) | EIN2 sense | EIN2 RNAi |
|---|---|---|
| >20 | 0 | 9 |
| 10-20 | 3 | 6 |
| 4-10 | 5 | 7 |
| ETR death | 0 | 12 |
| Total | 8 | 34 |

In contrast, none of the EIN2 sense flowers lasted more than 20 DAP. Similarly, 6% of the EIN2 RNAi lines contained flowers that lasted between 10 and 20 DAP, while only 3% of the EIN2 sense lines had flowers that lasted more than 10 days. Strong ethylene insensitivity has also been shown to increase the mortality rate of several plants, due to increased disease susceptibility (Knoester et al., 1998). Twelve percent of the RNAi lines exhibited premature death, likely due to strong ethylene insensitivity, while none of the EIN2 sense lines died prematurely. In total, 34% of the EIN2 RNAi lines exhibited increased ethylene insensitivity, compared to only 8% for the EIN2 sense lines. Ethylene treatment also indicated that the EIN2 RNAi flowers were less sensitive to ethylene than the EIN2 sense. For example, the EIN2 sense flowers lasted an average of 7 days after treatment, while the EIN2 RNAi flowers lasted 10-15 days (Table 2). Therefore the RNAi construct was more effective than EIN2 sense in reducing ethylene sensitivity and delaying flower senescence. Table 2: lists flower longevity in wildtype, etrl-1, EIN2 sense, and EIN2 RNAi flowers treated with ethylene. Detached flowers were treated for 16 hrs with 2-5 ppm ethylene. Wildtype flowers lasted almost 2 days after treatment, while the etr1-1 and EIN2 sense flowers lasted 7-8 days after treatment. The EIN2 RNAi flowers exhibited the greatest longevity, lasting from 9-15 days after treatment.

| Genotype | Days after treatment |
|---|---|
| WT | 1.6 + 0.0 |
| etr1-1 | 8.3 + 0.3 |
| EIN2 sense 1 | 6.6 + 0.3 |
| EIN2 sense 2 | 7.1 + 0.3 |
| EIN2 RNAi 2 | 9.7 + 2.7 |
| EIN2 RNAi 3 | 15.0 + 0.0 |

In addition to flower longevity, fruit ripening and adventitious root formation are also regulated by ethylene. The rate of fruit ripening and root formation was compared in wildtype and transgenic plants to determine whether RNAi-mediated suppression of PhEIN2 regulates these responses as well. Wild-type petunia fruit turns completely brown and the carpels dehisce when ripe. In this experiment wildtype fruit ripened 25 days after pollination, while fruit ripening was delayed in EIN2 sense and EIN2 RNAi lines by 27 and 31 days respectively. Ethylene-insensitive plants also exhibit reduced adventitious root formation (Clark et al., Plant Physiol 121: 53-59, 1999). To determine the role of EIN2 expression in adventitious root formation, propagation experiments were conducted on vegetative cuttings from wildtype and transgenic plants. Cuttings from EIN2 sense plants produced half the mass of adventitious roots compared to wild-type plants. Cuttings from EIN2 RNAi and etr1-1 plants produced almost no adventitious roots. (Table 3).

Table 3: shows the rate of fruit ripening and adventitious root formation in wildtype and ethylene-insensitive petunia plants. Fruit ripening was slower in the EIN2 RNAi plants than in etr1-1 and EIN2 sense plants. Since fruit ripening is regulated by ethylene, the slower ripening rate indicates a lower level of ethylene sensitivity. Adventitious root formation was greatly reduced in etr1-1 and EIN2 RNAi cuttings, further indicating that these lines had reduced ethylene sensitivity.

| | Fruit ripening | Root Growth | Root Dry Weight |
|---|---|---|---|
| Line | (Days) | Index | (g) |
| WT | 24.3 + 0.19 | 3.83 + 0.05 | 1.65 |
| EIN2 sense 2 | 26.8 + 0.43 | 3.42 + 0.32 | 0.88 |
| EIN2 RNAi 2 | 31.1 + 0.76 | 1.11 + 0.06 | <0.01 |
| etr1-1 | 26.4 + 0.30 | 1.56 + 0.32 | <0.01 |

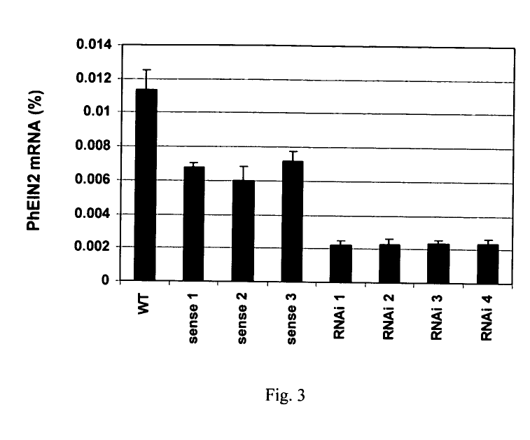

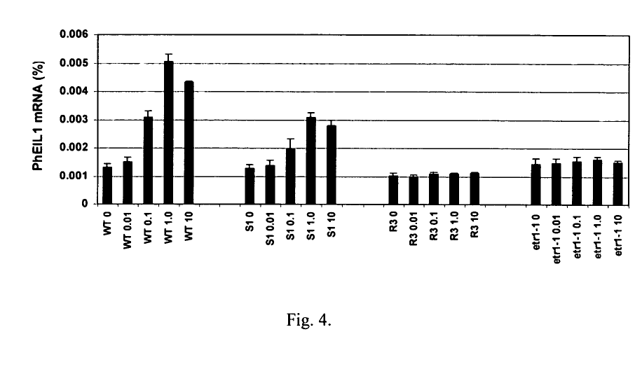

It is believed that this greater effectiveness of the RNAi construct was due to a higher level of cosuppression in the RNAi EIN2 plants. RNAi plants with the greatest flower longevity exhibited a 5-fold decrease in EIN2 expression, compared to a 2-fold decrease in EIN2 sense plants (FIG. 3). The greater decrease in ethylene sensitivity for the EIN2 RNAi plants was also indicated by the reduced expression of an ethylene-regulated gene. The expression of PhEILL1, a petunia homolog of the Arabidopsis EIN3 gene, is induced by exogenous ethylene in wildtype plants by up to 5-fold (FIG. 4). The maximum induction by exogenous ethylene in the EIN2 sense plants was reduced to approximately 2-fold, while the EIN2 RNAi plants exhibited no induction of PhEIL1 expression (FIG. 4). Therefore, although EIN2 sense plants were less sensitive to ethylene than wildtype plants, the RNAi construct was more effective in reducing ethylene sensitivity.

Although several different methods have been evaluated for manipulating ethylene response, these results show that cosuppression of the PhEIN2 gene through RNAi is one of the most effective means of manipulating ethylene sensitivity in petunia. RNAi-mediated EIN2 cosuppression produces a high percentage of transgenic lines with strong ethylene insensitivity and will permit the tissue-specific inhibition of ethylene responses that will be necessary for commercial application.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain sequences which are both related may be substituted for the agents described herein while the same or similar results would be achieved. All such similar substitutes and modifications apparent to those skilled in the art are deemed to be within the spirit, scope and concept of the invention as defined by the appended claims. For example, this method is useful in inhibiting gene expression in a cell. Preferably the present invention relates to the use of double stranded RNA (dsRNA) in inhibiting gene expression. In particular, it relates to inhibiting gene expression in plant cells and plants using dsRNA. Specifically, it relates to use of RNAi technology in inhibiting the expression of an ethylene-modulated gene. More specifically, it relates to use of the use of RNAi technology in inhibiting the expression of a floral specific gene in a plant, most specifically relates to the use of RNAi technology in inhibiting the expression of EIN2 gene in a plant and more importantly in the floral parts of a plant. More specifically, it relates to lengthen flower longevity in a plant. More specifically, the present invention relates to methods of altering the expression of genes in plants, in particular using RNA fragments of said genes, and to plants with altered gene expression obtained using the methods of the present invention. Thus the scope of the invention should be determined by the appended claims and their legal equivalents, rather than by the examples given.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks and other references (e.g., journal articles) that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); D. W. Mount, Bioinformatics: Sequence and Genome Analysis, Cold Spring Harbor Laboratory Press, Cold Spring Harbor, N.Y., 2000; Warren J. Ewens, Gregory R. Grant, Statistical Methods in Bioinformatics: An Introduction, Springer-Verlag, 2001 (ISBN: 0387952292); Pavel A. Pevzner, Computational Molecular Biology: An Algorithmic Approach, MIT Press, 2000 (ISBN: 0262161974); Peter Clote, Rolf Backofen, Computational Molecular Biology: An Introduction, John Wiley & Sons, Ltd., 2000 (ISBN: 0471872520). and the various references cited therein.

Equivalents

Those skilled in the art will recognize, or be able to ascertain using no more than routine experimentation, many equivalents to the specific embodiments of the invention described herein. Such equivalents are intended to be encompassed by the following claims.

1. A method for inhibiting the expression of a target gene in a plant cell, the method comprising introducing into the plant cell an RNA comprising a double stranded structure having a nucleotide sequence which is substantially identical to at least a part of the target gene, wherein the target gene is an ethylene signaling gene.

2. A method as claimed in claim 1, wherein the target gene is an endogenous gene.

3. A method as claimed in claim 1, wherein the target gene is a tissue specific gene.

4. A method as claimed in claim 1, wherein the target gene is a floral tissue specific gene.

5. A method as claimed in claim 1, 2, 3 or 4, wherein the RNA is produced outside the cell.

6. A method as claimed in claim 5, wherein the RNA is produced outside the cell, and is then injected into the cell.

7. A method as claimed in claim 1, 2, 3 or 4, wherein the RNA is produced within the cell.

8. A method as claimed in claim 1, 2, 3 or 4, wherein the RNA is produced outside the cell, wherein the RNA is produced recombinantly.

9. A method as claimed in claim 1, 2, 3 or 4, wherein the RNA is produced outside the cell recombinantly and is then injected into the cell.

10. A method as claimed in claim 1, 2, 3 or 4, wherein the RNA is recombinantly produced within the cell.

11. A method as claimed in claim 1, 2, 3 or 4, wherein the RNA is produced within the cell using an expression vector in the cell.

12. A method as claimed in claim 1, 2, 3 or 4, wherein the RNA is recombinantly produced outside the cell using an expression vector in the cell.

13. A method as claimed in any one of claims 1, 2, 3 or 4, wherein the RNA comprises a single self-complementary RNA strand.

14. A method as claimed in any one of claims 1, 2, 3 or 4, wherein the RNA comprises two separate complementary RNA strands.

15. A method as claimed in any one of claims 1, 2, 3 or 4, wherein the nucleotide sequence is substantially identical to the whole of the target gene.

16. A method as claimed in any one of claims 1, 2, 3 or 4, wherein the nucleotide sequence has 90%, 95% or 100% identity with at least a part of the target gene.

17. A method as claimed in any one of claims 1, 2, 3 or 4, wherein the target gene affects or is likely to affect flowering.

18. A method as claimed in any one of claims 1, 2, 3 or 4, wherein the target gene affects or is likely to affect flowering longevity.

19. A method as claimed in any one of claims 1, 2, 3 or 4, wherein the cell is a plant cell.

20. A method as claimed in any one of claims 1, 2, 3 or 4, wherein the plant cell is an embryogenic or non embryogenic cell.

21. A method as claimed in any one of claims 1, 2, 3 or 4, wherein the plant cell is a cell growing in a tissue culture medium.

22. A chimeric gene comprising a promoter active in plants operatively linked to the nucleotide sequence of claim 1.

23. A recombinant vector comprising the chimeric gene of claim 22.

24. A host cell comprising the chimeric gene of claim 22.

25. A plant comprising the chimeric gene of claim 22.

26. A transgenic plant comprising the chimeric gene of claim 22.

27. A seed from the plant of claim 26.

28. A method for inhibiting the expression of a target gene in a cell, the method comprising introducing into the cell an RNA comprising a double stranded structure having a nucleotide sequence which is substantially identical to at least a part of the target gene, wherein the target gene is an EIN gene.

29. A method for inhibiting the expression of a target gene in a cell, the method comprising introducing into the cell an RNA comprising a double stranded structure having a nucleotide sequence which is identical to at least a part of the target gene, wherein the target gene is an EIN gene.

30. An expression construct, the construct coding for an RNA which forms a double stranded structure having a nucleotide sequence which is substantially identical to at least a part of a target gene and which is derived from a template, wherein the target gene is an ethylene signaling gene.

31. A cell containing an expression construct, the construct coding for an RNA which forms a double stranded structure having a nucleotide sequence which is substantially identical to at least a part of a target gene and which is derived from a template, wherein the target gene is an ethylene signaling gene.

32. A composition comprising RNA which comprises a double stranded structure having a nucleotide sequence which is substantially identical to at least a part of a target gene in a cell and which is derived from a template together with an acceptable carrier; wherein the target gene is an ethylene signaling gene.

33. A kit for inhibiting expression of a target gene in cell, the kit comprising: RNA which comprises a double stranded structure having a nucleotide sequence which is substantially identical to at least a part of a target gene in cell and which is derived from template; and a vehicle that promotes introduction of the RNA to the cell, wherein the target gene is an ethylene signaling gene.

34. A method for inhibiting the expression of a target gene in a cell, the method comprising: introducing into the cell an RNA comprising a double stranded structure having a nucleotide sequence which is substantially identical to at least a part of the target gene and which is derived from an endogenous template, wherein the target gene is an ethylene signaling gene.

35. A method for inhibiting the expression of a target gene in a cell, the method comprising: introducing into the cell an RNA comprising a double stranded structure having a nucleotide sequence which is substantially identical to at least a part of the target gene; and verifying inhibition of expression of the target gene, wherein the wherein the target gene is an ethylene signaling gene or is likely to be regulated by ethylene.

36. A method for inhibiting the expression of a target gene in a cell, the method comprising: introducing into the cell an RNA comprising a double stranded structure having a nucleotide sequence which is substantially identical to at least a part of the target gene and which is derived from an endogenous template, wherein the target gene is an ethylene signaling gene or is likely to be regulated by ethylene.

37. A method for inhibiting the expression of a target gene in a cell, the method comprising: introducing into the cell an RNA comprising a double stranded structure having a nucleotide sequence which is substantially identical to at least a part of the target gene and which is derived from an endogenous template; and verifying inhibition of expression of an ethylene signaling gene, wherein the wherein the target gene is regulated by ethylene.

38. An RNA comprising a double stranded structure having a nucleotide sequence which is substantially identical to at least a part of a target gene in a cell and which is derived from a template for use as a mean for affecting ethylene response in a cell.

39. An RNA comprising a double stranded structure having a nucleotide sequence which is substantially identical to at least a part of a target gene in a cell and which is derived from an endogenous or an exogenous template for use as a mean for affecting ethylene response in a cell.

40. A method to inhibit expression of a target gene comprising: (a) providing an organism containing a target cell, wherein the target cell contains the target gene and the target gene is expressed in the target cell; (b) contacting the organism with a ribonucleic acid (RNA), wherein the RNA is comprised of a double-stranded structure with duplexed ribonucleic acid strands and one of the strands is able to duplex with a portion of the target gene; and
(c) introducing the RNA into the target cell, thereby inhibiting expression of the target gene; wherein the target gene is modulated by ethylene.

41. A method for inhibiting the expression of a target gene in a cell, the method comprising introducing into the cell an RNA comprising a double stranded structure having a nucleotide sequence which is substantially identical to at least a part of the target gene, wherein the target gene is an EIN2 gene.

42. A method for inhibiting the expression of a target gene in a cell, the method comprising introducing into the cell an RNA comprising a double stranded structure having a nucleotide sequence which is identical to at least a part of the target gene, wherein the target gene is an EIN2 gene.

43. A method for inhibiting the expression of a target gene in a cell, the method comprising introducing into the cell an RNA comprising a double stranded structure having a nucleotide sequence which is substantially identical to at least a part of the target gene, wherein the target gene is an EIN3 gene.

44. A method for inhibiting the expression of a target gene in a cell, the method comprising introducing into the cell an RNA comprising a double stranded structure having a nucleotide sequence which is identical to at least a part of the target gene, wherein the target gene is an EIN2 gene.


SEQUENCE LISTING (4 sequences)

SEQ ID NO: 1 - DNA, 22 bp, Petunia sp.
  primer_bind (1)..(22): PhEIN2 forward primer
tgtgtttttctggctgcaactg

SEQ ID NO: 2 - DNA, 21 bp, Petunia sp.
  primer_bind (1)..(21): PhEIN2 reverse primer
gcactgccattgtccaagaga

SEQ ID NO: 3 - DNA, 26 bp, Petunia sp.
  primer_bind (1)..(26): PhEIL1 forward primer
gccttttccatctccacttactattg

SEQ ID NO: 4 - DNA, 23 bp, Petunia sp.
  primer_bind (1)..(23): PhEIL1 reverse primer
tggatatcaagccccaaattaaa